(19) United States
(12) Patent Application Publication
(10) Pub. No.: US 2003/0017966 A1
(43) Pub. Date: Jan. 23, 2003

(54) MC4-R as target for the identification of compounds used to treat drug addiction

(75) Inventor: Ronald Duman, Guilford, CT (US)

Correspondence Address: ANITA L. MEIKLEJOHN, PH.D., Fish & Richardson P.C., 225 Franklin Street, Boston, MA, 02110-2804, US

(73) Assignee: Yale University, a Connecticut corporation

(21) Appl. No.: 10/226,594

(22) Filed: Aug. 23, 2002

Related U.S. Application Data
(63) Continuation of application No. 09/981,698, filed on Oct. 17, 2001.
Application No. 09/981,698 is a continuation of application No. 09/385,763, filed on Aug. 30, 1999, now abandoned.
(60) Provisional application No. 60/099,104, filed on Sep. 3, 1998.

(51) Int. Cl. A61K 31/00; G01N 33/53

(52) U.S. Cl. 514/1; 435/7.1

(57) ABSTRACT

The present invention relates to drug screening assays and therapeutic methods for the treatment of addictive behavior disorders, such as cocaine and morphine addiction utilizing the melanocortin 4-receptor (MC4-R) as the target for intervention. The invention also relates to compounds that antagonize the activity or expression of the MC4-R, and the use of such compounds in the treatment of addictive behavior disorders.

### FIELD OF THE INVENTION

The present invention is in the field of drug discovery to treat addictive behavior, particularly drug addiction. The present invention specifically provides drug screening assays and therapeutic methods for the treatment of addictive behavior, particularly drug addiction, involving the melanocortin 4-receptor (MC4-R). The invention also provides novel methods of using antagonists of the activity or expression of MC4-R to treat addictive disorders.

### BACKGROUND OF THE INVENTION

Drug Addiction

It is well known that the chronic administration of opioids, cocaine and other drugs of abuse results in tolerance and, eventually, dependence. The use of cocaine, opiates, and alcohol are extremely widespread in many countries, despite the well known adverse effects of their use. Drug abuse endures as one of the major public health problems in the United States, and throughout the world. One of the core features of addictive disorders, in laboratory animals as well as in humans, is that drugs of abuse are acutely reinforcing and produce intense drug craving following chronic exposure.

Behavioral and pharmacological studies have implicated the mesolimbic dopamine system (containing the ventral tegmental area [VTA] and its projections, e.g., the nucleus accumbens [NAc]) in the acute reinforcement and craving seen with opiates, cocaine, alcohol, and other drugs of abuse. An important goal of research in this area is to identify changes in this neural pathway that are caused by rugs of abuse and account for the intense craving seen with chronic drug use. Another critical goal is the identification of factors that can inhibit or reverse these changes to the neural pathway.

Over the past several years, studies have identified a series of common and specific actions of opiates, cocaine, and alcohol on the mesolimbic dopamine system (see, Nestler et al., 1993, Neuron 11:995-1006). Different classes of neuronal receptors and neurotransmitters in the brain have been implicated in the complex mechanisms underlying, for example, the addictive effects of opiates. Experimental findings suggest that the opioid, dopaminergic, serotonergic, and benzodiazepine receptor subtypes are involved in addictive effects.

In the VTA, the effects of various drugs of abuse include increased levels of tyrosine hydroxylase (TH) and glial fibrillary acidic protein (GFAP), and decreased levels of neurofilaments. In the NAc, the effects include decreased levels of the inhibitory G protein, Gi alpha, and increased levels of adenylyl cyclase and cyclic AMP-dependent protein kinase. Increasing direct evidence now supports a role for these various biochemical adaptations in the behavioral actions of drugs of abuse mediated via the mesolimbic dopamine system. The finding of increased levels of glial filaments, and reduced levels of neurofilaments, in the VTA suggests that a major form of plasticity, perhaps even neural injury, occurs in this brain region during the course of chronic drug exposure. This possibility is further supported by the observation that chronic morphine or cocaine administration reduces axoplasmic transport, specifically from the VTA to the NA.

Physical dependence or drug addiction to drugs such as narcotics or cocaine has been traditionally treated by drug withdrawal through withholding the drug from the drug dependent individual, gradually decreasing the amount of drug, particularly with opioids, taken by the individual over time, administering an antagonistic drug, or substituting another drug, such as methadone, buprenorphine, or methadyl acetate, for the drug to ameliorate the physical need for the drug. When an drug is discontinued, withdrawal symptoms appear, the character and severity of which are dependent upon such factors as the particular drug being withdrawn, the daily dose of the drug that is being withdrawn, the duration of use of the drug, and the health of the drug dependent individual.

It is therefore desirable to identify the biological mechanism of drug addiction, and addictive behavior in general, so that better and more effective therapeutic compounds can be identified. The present invention identifies one of the mechanisms of drug addiction and provides methods of identifying compounds for use in Greasing drug addiction.

Melanocortin Receptors

Melanocortins, products of pro-opiomelanocortin (POMC) post-translational processing, are known to have a broad array of physiological actions. Nakanishi et al., Nature 278:423-427 (1979). Aside from their well known effects on adrenal cortical functions (adrenocorticotropic hormone, ACTH) and on melanocytes (melanocyte stimulating hormone, MSH), melanocortins have been suggested to affect behavior, learning, memory, control of the cardiovascular system, analgesia, thermoregulation, body weight and the release of other neurohumoral agents including prolactin, luteinizing hormone, and biogenic amines. De Wied et al., Methods Achiev. Exp. Pathol. 15:167-199 (1991); De Wied et al., Physiol. Rev. 62:977-1059 (1982); Gruber et al., Am. J. Physiol. 257:R681-R694 (1989); Murphy et al., Science 210:1247-1249 (1980); Murphy et al., Science 221:192-193 (1983); Ellerkmann et al., Endocrinol. 130:133-138 (1992) and Versteeg et al., Life Sci. 38:835-840 (1986). Peripherally, melanocortins have been suggested to have immunomodulatory and neurotrophic properties and to be involved in events surrounding parturition. Cannon et al., J. Immunol. 137:2232-2236 (1986); Gispen, Trends Pharm. Sci. 11:221-222 (1992); Wilson, J. F., Clin. Endocrinol. 17:233-242 (1982); Clark et al., Nature 273:163-164 (1978) and Silman et al., Nature 260:716-718 (1976). Furthermore, melanocortins are present in a myriad of normal human tissues including the brain, adrenal, skin, testis, spleen, kidney, ovary, lung, thyroid, liver, colon, small intestine and pancreas. Tatro et al., Endocrinol. 121:1900-1907 (1987); Mountjoy et al., Science 257:1248-1251 (1992); Chhajlani et al., FEBS Lett. 309:417-420 (1992); Gantz et al. J. Biol. Chem. 268:8246-8250 (1993) and Gants et al., J. Biol. Chem. 268:15174-15179 (1993).

Recent studies have described a diversity of subtypes of receptors for the melanocortin peptides and determined that they all belong to the superfamily of seven transmembrane G-protein linked cell surface receptors. Mountjoy et al., Science 257:1248-1251 (1992); Chhajlani et al., FEBS Lett. 3:417-420 (1992); Gantz, Biol. Chem. 268:8246-8250 (1993). In total five receptors have been identifed by sequence homology to the first cloned receptor.

The first two melanocortin receptors cloned were the melanocyte MSH receptor, MC1-R, and the adrenocortical ACTH receptor, MC2-R (Mountjoy, Science 257:1248-1251 (1992); Chhajlani & Wikberg, FEBS Lett. 309:417-420 (1992)). Subsequently, three additional melanocortin receptor genes were cloned that recognize the core heptapeptide sequence (MEHFRWG) of melanocortins. Two of these receptors have been shown to be expressed primarily in the brain, MC3-R (Roselli-Rehfuss et al., Proc. Natl. Acad. Sci. USA 90:8856-8860 (1993); Gantz et al., J. Biol. Chem. 268:8246-8250 (1993)) and MC4-R (Gantz et al., J. Biol. Chem. 268:15174-15179 (1993); Mountjoy et al., Mol. Endo. 8:1298-1308 (1994)). A fifth melanocortin receptor (originally called MC2-R) is expressed in numerous peripheral organs as well as the brain (Chhajlani et al., Biochem. Biophys. Res. Commun. 195:866-873 (1993); Gantz et al., Biochem. Biophs. Res. Commun. 200:1214-1220 (1994)). The native ligands and functions of these latter three receptors remains largely unknown.

Recently, the Agouti protein in mice (U.S. Pat. No. 5,789,651), and the agouti-related protein in humans (U.S. Pat. No. 5,766,877), were shown to be natural ligands for the MC4-R protein. The agouti protein is a secreted protein expressed in hair follicles and the epidermis, the expression of which correlates with the synthesis of the yellow pigment associated with the agouti phenotype (Miller et al., Gene & Development 7:454-467 1993)). Certain dominant mutations of the agouti gene result in de-regulated, ubiquitous expression of the agouti protein in mice, demonstrating pleiotropic effects that include obesity and increased tumor susceptibility. (Miller et al., supra, (1993); Michaud et al., Genes & Development 7:1203-1213 (1993)). Ectopic expression of the wild-type agouti protein in transgenic mice results in obesity, diabetes, and the yellow coat color commonly observed in spontaneous obese mutants (Klebig, et al., Proc. Natl. Acad. Sci. USA 92:4728-4732 (1995)). For reviews, see Jackson, Nature 362:587-588 (1993); Conklin & Bourne, Nature 364:110 (1993); Siracusa, TIG 10:423-428 (1994); Yen et al., FASEB J. 8:479-488 (1994); Ezzell, J. NIH Res. 6:31-33 (1994); and Manne et al., Proc. Sci. USA 92:4721-4724 (1995). Agouti has been reported to be a competitive antagonist of (MSH binding to the MC1-R and MC4-R in vitro (Lu et al., Nature 371:799-802 (1994)), and the authors speculated that ectopic expression of agouti may lead to obesity by antagonism of melanocortin receptors expressed outside the hair follicle. In this regard, a number of theories have been proposed to account for the induction of obesity by ectopic expression of agouti.

While prior structure-function analyses have been reported in the past on the affinity and potency of the alpha -MSH peptide at the MSH receptor site (for reviews see Peptide Protein Rev 3:1 (1984), The Melanotropic Peptides, Vol. I, II, and III (CRC Press) (1988)), only a few relatively weak antagonists have resulted from these studies [see Int J Peptide Protein Res 35:228 (1990); Peptides 11:351 (1990); and Peptide Res 3:140 (1989)]. However, these papers demonstrate that methods for identifying anatgonists of the melanocortin family of receptors is within the skill of the art.

### SUMMARY OF THE INVENTION

The present invention is based on several sets of experiments that includes the observation that mice deficient in the MC4-R protein (MC4-R knockout mice) do not display the addictive-effects induced by drugs of addiction, particularly cocaine and morphine. Based on these observations, the present invention provides a novel target for the identification and development of compounds that can be used to treat addictive behavior, particularly drug addiction as well a therapeutic target for treating drug addition.

Specifically, assays are provided (and equivalent assays can be configured) to identify antagonists of the MC4-R receptor protein, in particular receptor binding assays, competition binding assays, activity assays, expression assays (transcription and translation), animal assays and combinations thereof. Such assays result in the identification of compounds that can be used in treating addictive disorders.

It is important to note that assays for identifying antagonists of the MC4-R protein and assays for testing a compound's ability to modulate an addictive behavior in an animal are known in the art.

### BRIEF DESCRIPTION OF THE DRAWINGS

FIG. 1. Deduced amino acid sequences of the melanocortin receptors. The serpentine structure of the melanocortin receptors predicts that the hydrophilic domains located between the TM domains are arranged alternately outside and within the cell to form extracellular domains (ECD; amino acid residues 1-74, 137-155, 219-231 and 305-316 in FIG. 1) and cytoplasmic domains (CD; amino acid residues 102-112, 178-197, 251-280 and 339-end in FIG. 1) of the receptor. The predicted transmembrane domains are denoted by overbars and Roman numerals, and the four extracellular domains (ECD1, ECD2, ECD3 and ECD4) and four cytoplasmic domains (CD1, CD2, CD3 and CD4) are indicated.

FIG. 2. Regional distribution of MC4-R mRNA expression in the rat brain.

FIG. 3. Regional specific expression of MC4-R mRNA in the rat brain

FIG. 4. Changes seen in MC4-R mRNA levels in regional centers of the rat brain caused by the administration of cocaine.

FIG. 5. Changes seen in MC4-R mRNA levels in the neostriatum of the rat brain caused by the administration of varying doses of morphine.

FIG. 6. Changes seen in POMC mRNA levels in regional centers of the rat brain caused by the administration of cocaine.

FIG. 7. Changes seen in (α-MSH-induced grooming behavior in rats caused by the administration of combinations of saline (sal), (α-MSH, and cocaine (coc).

FIG. 8. Changes seen in (α-MSH-Induced locomotor activity in rats caused by the administration of combinations of saline (sal), (α-MSH, and cocaine (coc).

FIG. 9. Cocaine (10 mg/kg) induced locomotor sensitization in Agouti and C57 mice.

FIG. 10. Novelty induced locomotor activity in Agouti and C57 mice.

FIG. 11. Cocaine (5 mg/kg) induced locomotor sensitization in Agouti and C57 (wild-type) mice.

FIG. 12. Cocaine (10 mg/kg) induced locomotor sensitization in C57 (wild-type) and MC4-R knockout (homozygous) mice.

FIG. 13. Cocaine (1 mg/kg) induced locomotor sensitization in C57 (wild-type) and MC4-R knockout (homozygous) mice.

FIG. 14. Baseline locomotor activity in C57 (wild-type) and MC4-R knockout (homozygous and heterozygous) mice.

FIG. 15. Cocaine (10 mg/kg) induced locomotor sensitization in rats treated with and without Intra-Nac infusions of SHU9119 (an antagonist of MC4-R).

FIG. 16. Cocaine induced place preference in rats treated with and without Intra-Nac-infusions of SHU9119 (an antagonist of MC4-R).

### DESCRIPTION OF THE PREFERRED EMBODIMENTS

MC4-R and Addiction

Described below are experiments demonstrating that some of the addictive effects of drugs of addiction (e.g., cocaine and morphine) are potentiated, in part, by the activity of the melanocortin-4 receptor (MC4-R). Mice lacking MC4-R (MC4-R knockout mice) do not demonstrate behavioral responses indicative of drug addiction induced to chronic and acute administration of cocaine or morphine. Based on these observations, one aspect of the present invention provides one of the specific molecular mechanisms that mediates addictive behavior.

As described below, this molecular mechanism can be used: 1) to identify and isolate compounds for treating drug addiction; 2) as a target to rationally design compounds for use in treating drug addiction; and 3) as a therapeutic target for treating drug addiction.

Methods To Identify Compounds For Treating Drug Addiction

The present invention provides methods for identifying compounds that can be used to treat drug addiction. These methods are based on identifying antagonists of the melanocortin-4 receptor (MC4-R).

In general, three formats used in drug discovery, cell based systems, cell free systems, and animal based systems, can be adapted for use in the present invention. Further, the assays of the present invention can be configured as binding assays, competitive binding assays or activity-based assays. Lastly, a combination of cell free, cell based, and animal based assays can be used.

Specifically, to identify a therapeutic compound for use in treating drug addiction using a cell based binding assay, a cell expressing a MC4-R protein, a cell expressing a fragment of the MC4-R protein, or a cell expressing a protein containing a fragment of the MC4-R protein as a fusion protein (hereinafter collectively ôa cell expressing the MC4-R proteinö) is incubated in the presence and absence of a compound to be tested. After mixing under conditions that allow association of the MC4-R protein with the compound (if such an interaction will occur), the two mixtures are analyzed and compared to determine if the compound bound to the cell expressing the MC4-R protein. Compounds that bind to cells expressing the MC4-R protein will be identified as a potential antagonist of MC4-R. Preferred potential antogonists do not substantially bind to otherwise identical cells which do not express MC4-R.

To identify a therapeutic compound for use in treating drug addiction using a cell free binding assay, an isolated MC4-R protein, an isolated fragment of the MC4-R protein, or an isolated protein containing a fragment of the MC4-R protein as a fusion protein (hereinafter collectively the MC4-R-proteino) is incubated in the presence and absence of a compound to be tested. After mixing under conditions that allow association of the MC4-R protein with the compound (if such an interaction will occur), the two mixtures are analyzed and compared to determine if the compound bound to the MC4-R protein. Compounds that bind to the MC4-R protein will be identified as a potential antagonist of MC4-R.

To identify a therapeutic compound for use in treating drug addiction using a cell based competition binding assay, a cell expressing a MC4-R protein, a cell expressing a fragment of the MC4-R protein, or a cell expressing a protein containing a fragment of the MC4-R protein as a fusion protein (hereinafter collectively ôa cell expressing the MC4-R proteino) is incubated in the presence and absence of a compound to be tested and further in the presence of a ligand of MC4-R. After mixing under conditions that allow association of the cell expressing the MC4-R protein with the MC4-R ligand, the two mixtures are analyzed and compared to determine if the compound reduced or blocked the binding of the MC4-R ligand to the cell expressing the MC4-R protein. Compounds that reduce or block the binding of the MC4-R ligand to the cell expressing the MC4-R protein will be identified as a potential antagonist of MC4-R.

To identify a therapeutic compound for use in treating drug addiction using a cell-free based competition binding assay, a MC4-R protein, a fragment of the MC4-R protein, or a protein containing a fragment of the MC4-R protein as a fusion protein (hereinafter collectively ôa MC4-R proteinö) is incubated in the presence and absence of a compound to be tested and further in the presence of a ligand of MC4-R. After mixing under conditions that allow association of the MC4-R protein with the MC4-R ligand, the two mixtures are analyzed and compared to determine if the compound reduced or blocked the binding of the MC4-R ligand to the MC4-R protein. Compounds that reduce or block the binding of the MC4-R ligand to the MC4-R protein will be identified as a potential antagonist of MC4-R.

To identify a therapeutic compound for use in treating drug addiction using a cell based activity assay, a cell expressing a MC4-R protein, a cell expressing a functional fragment of the MC4-R protein, or a cell expressing a protein containing a functional fragment of the MC4-R protein as a fusion protein (hereinafter collectively a cell expressing the MC4-R protein) is incubated in the presence and absence of a compound to be tested. After mixing under conditions that allow association of the MC4-R protein with the compound (if such an interaction will occur), the activity of the MC4-R protein is determined in the two mixtures and compared to determine if the compound antagonized the MC4-R activity in the cell. Compounds that antagonize the activity of the MC4-R protein will be identified as a potential antagonist of MC4-R for use in treating drug addiction. Such activity assays are best performed in the presence of a ligand of MC4-R.

Each of the methods outlined above will be discussed in greater detail below.

The methods of the present invention are suitable for use in identifying compounds for treating addiction to a wide variety of addictive compounds. Repeated administration to a subject of certain drugs such as cocaine, opiates, alcohol, hallucinogens, minor tranquilizers, nicotine, and stimulants can lead to physical and/or psychological dependence upon that drug or substance. Although almost any drug is capable of addiction, certain drugs demonstrated a marked propensity to become addictive. These include opiates (opium, morphine, heroin, and so called “designer drugs,” which are opiates that have been chemically modified to avoid literal violation of the controlled substance laws, or to create a better or different psychophysiological effect), methadone, cocaine, nicotine, alcohol, certain depressants, and certain stimulants. When the drug or substance of abuse is withdrawn from a dependent subject, the subject develops certain symptoms including sleep and mood disturbance and intense craving for the drug or substance of abuse. These symptoms may be collectively described as a withdrawal syndrome in connection with the present invention. As discussed in the Background, many drugs of addiction have been found to stimulate similar behavior centers of the brain. Experimental results described in the Examples (below) demonstrate that two classes of addictive compounds, cocaine and opiates, stimulate similar biological response, e.g. increased MC4-R expression in the nucleus accumbens and neostriatum. Accordingly, compounds identified in the present methods will be useful in treating addictive behavior to a wide variety of addictive stimulus.

As used herein, the term “addictive disease, disorder, behavior or addiction” refers to a disease or disorder in which the subject has an extreme craving or compulsion to repeat a particular behavior. The present invention is particularly directed to therapeutic treatment of a drug addiction. Notwithstanding the specifically exemplified ability of the present invention to modulate the biochemistry and behavior that correlate with drug abuse, the invention further extends to the treatment of addictive psychological diseases or disorders, such as, but not limited to, obsessive-compulsive disease.

The methods of the present invention and the compounds identified using the present methods can be used to treat or prevent an addictive disease or disorder in a subject. Preferably the subject is a human, however, as animals in addition to humans may demonstrate addictive diseases or disorders, whether resulting from addiction to opiates or other drugs subsequent to a veterinary procedure or as a result of a psychological disorder, such as an obsessive compulsive-type of disorder, the invention can be used in birds, such as chickens, turkeys, and pets; in mammals, including but not limited to domesticated animals (canine and feline); farm animals (bovine, ovine, equine, caprine, porcine, and the like); rodents; and undomesticated animals.

The MC4-R protein used in the present method can either be the known human MC4-R receptor (Mol. Endocrinol. 8:1298-1308 (1994), U.S. Pat. No. 5,703,220, Genbank Accession No. 998457), any allelic variant thereof (Genbank Accession No. 417280 or 136281), or any ortholog of the human MC4-R protein, such as, but not limited to mouse MC4-R (Genbank Accession No. 3024117) and rat MC4-R (Genbank Accession No. 2494982). Since members of the MC4-R family of proteins display high levels of sequence homology, most members of this family can be used interchangeably in the present methods.

As indicated above, as an alternative to the entire MC4-R protein, a fragment of the MC4-R protein can be used. Such fragments may be selected based on a function, such as the ligand or G-protein binding domain, or can be randomly generated. Preferred fragments will contain the ligand binding domain of the MC4-R protein.

Alternatively, a fusion protein containing the MC4-R protein, or a fragment thereof, can be used. The use of a fusion protein in compound screening assays is well known in the art since fusion protein can aid in configuring such assays, for example with the use of an IgG fusion protein to aid in immobilization.

The MC4-R used in the present method can be any isolated member of the MC4-R ligand family. Examples of MC4-R ligands include human (α-MSH (FEBS Lett. 135:97-102 (1981), Science 2-7:543 (1992), Genbank Accession No. P01189), murine (α-MSH (FEBS Lett. 156:67-71 (1983), Genbank Accession No. P01193), rat (α-MSH (FEBS Lett. 193:54-58 (1985), Genbank Accession No. P01194), the agouti protein (U.S. Pat. No. 5,789,651), and the recently clone agouti related protein from humans (U.S. Pat. No. 5,766,877).

The assays described herein are intended to identify compounds that affect MC4-R activity. For example, compounds that affect MC4-R activity include but are not limited to compounds that bind to MC4-R, inhibit binding of a natural ligand, block activation, and compounds that bind to the natural ligand of the MC4-R and reduce ligand activity. Compounds that affect MC4-R gene activity (by affecting MC4-R gene expression, including molecules, e.g., proteins or small organic molecules, that affect transcription or interfere with splicing events so that expression of the full length or the truncated form of the MC4-R can be antagonized) can also be identified using the screen assays of the invention. However, it should be noted that the screening assays described can also identify compounds that antagonize MC4-R signal transduction (e.g., compounds which affect downstream signaling events, such as inhibitors or enhancers of one or more G protein activities which participate in transducing the signal induced by ligand binding to the MC4-R). The identification and use of compounds that affect signaling events downstream of MC4-R and thus modulate effects of MC4-R on the development of addictive behavior disorders are within the scope of the invention.

The compounds that may be screened in accordance with the assays of the invention include, but are not limited to, peptides, antibodies and fragments thereof, and other small molecules or organic compounds that bind to the MC4-R and inhibit the activity triggered by the natural ligand (i.e., antagonists) and peptides, antibodies or fragments thereof, and other small molecule or organic compound that bind to and “neutralize” the natural ligand.

Compounds may include, but are not limited to, peptides such as, for example, soluble peptides, including but not limited to members of random peptide libraries; (see, e.g., Lam et al., 1991, Nature 354:82-84; Houghten et al., 1991, Nature 354:84-86), and combinatorial chemistry-derived molecular library made of D- and/or L- configuration amino acids, phosphopeptides (including, but not limited to, members of random or partially degenerate, directed phosphopeptide libraries; see, e.g., Songyang et al., 1993, Cell 72:767-778), antibodies (including, but not limited to, polyclonal, monoclonal, humanized, anti-idiotypic, chimeric or single chain antibodies, and FAb, F(ab( )₂ and FAb expression library fragments, and epitope-binding fragments thereof), and small organic or inorganic molecules.

Other compounds that can be screened in accordance with the invention include, but are not limited to, small organic molecules that are able to cross the blood-brain barrier, gain entry into an appropriate cell and affect the expression of the MC4-R gene or some other gene involved in the MC4-R signal transduction pathway (e.g., by interacting with the regulatory region or transcription factors involved in gene expression) and compounds that affect the activity of the MC4-R or the activity of some other intracellular factor involved in the MC4-R signal transduction pathway, such as, for example, the MC4-R associated G protein.

Computer modeling and searching technologies permit identification of compounds, or the improvement of already identified compounds, that can antagonize MC4-R expression or activity. Having identified such a compound or composition, the active sites or regions are identified. Such active sites might typically be ligand binding sites. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found.

Next, the three dimensional geometric structure of the active site is determined. This can be done by known methods,-including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the active site structure determined.

If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, whether experimentally, by modeling, or by a combination, candidate modulating compounds can be identified by searching databases containing compounds along with information on their molecular structure. Such a search seeks compounds having structures that match the determined active site structure and that interact with the groups defining the active site. Such-a search can be manual, but is preferably computer assisted. These compounds found from this search are potential MC4-R antagonizing compounds.

Alternatively, these methods can be used to identify improved antagonizing compounds from an already known antagonizing compound or ligand. The composition of the known compound can be modified and the structural effects of modification can be determined using the experimental and computer modeling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

Further experimental and computer modeling methods useful to identify modulating compounds based upon identification of the active sites of MC4-R and related transduction and transcription factors will be apparent to those of skill in the art.

Examples of molecular modeling systems are the CHARMm and QUANTA programs (Polygen Corporation, Waltham, Mass.). CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al. (1988, Acta Pharmaceutical Fennica 97:159-166); Ripka (1988 New Scientist 54-57); McKinaly and Rossmann (1989, Annu. Rev. Pharmacol. Toxiciol. 29:111-122); Perry and Davies, (OSAR: Quantitative Structure-Activity Relationships in Drug Design pp.á189-193 Alan R. Liss, Inc. 1989); Lewis and Dean (1989, Proc. R. Soc. Lond. 236:125-140 and 141-162); and, with respect to a model receptor for nucleic acid components, Askew et al. (1989, J. Am. Chem. Soc. 111:1082-1090). Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific to regions of DNA or RNA, once that region is identified.

Although described above with reference to design and generation of compounds that could alter binding, one can also screen libraries of known compounds, including natural products or synthetic chemicals, and biologically active materials, including proteins, for compounds that are antagonists of MC4-R.

Cell-Based Assays

In accordance with the invention, a cell-based assay system can be used to screen for compounds that antagonize the activity of the MC4-R and thereby, modulate addictive behavior. To this end, cells that endogenously express MC4-R can be used to screen for compounds. Alternatively, cell lines, such as 293 cells, COS cells, CHO cells, fibroblasts, and the like, genetically engineered to express the MC4-R can be used for screening purposes. Preferably, host cells genetically engineered to express a functional receptor that responds to activation by melanocortin peptides can be used as an endpoint in the assay (as measured by, e.g., a chemical, physiological, biological, or phenotypic change, induction of a host cell gene or a reporter gene, change in cAMP levels, adenylyl cyclase activity, host cell G protein activity, extracellular acidification rate, host cell kinase activity, proliferation, differentiation, etc.) In addition, cell-based assay systems can be used to screen for compounds that antagonize the activity of a mutant MC4-R and thereby, modulate addictive behavior. For example, compounds may be identified that increase the activity of mutant MC4-R thereby alleviating the symptoms of addictive behavior disorders arising from mutant MC4-R. Cell lines, such as 293 cells, OS cells, CHO cells, fibroblasts and the like may be genetically engineered to express mutant receptor. Alternatively, cells that endogenously express mutant MC4 receptor can be used to screen for compounds.

To be useful in screening assays that detect receptor activity, the host cells expressing functional MC4-R should give a significant response to MC4-R ligand, preferably greater than 5-fold induction over background. Host cells should preferably possess a number of characteristics, depending on the readout, to maximize the inductive response by melanocortin peptides, for example, for detecting a strong induction of a cAMP response element (CRE) reporter gene: (a) a low natural level of cAMP, (b) expression of G proteins capable of interacting with the MC4-R, (c) a high level of adenylyl cyclase, (d) a high level of protein kinase A, (e) a low level of phosphodiesterases, and (f) a high level of cAMP response element binding protein would be advantageous. To increase response to melanocortin peptide, host cells can be engineered to express a greater amount of favorable factors or a lesser amount of unfavorable factors in addition, alternative pathways for induction of the CRE reporter could be eliminated to reduce basal level expression.

In utilizing such cell systems, the cells expressing the melanocortin receptor are exposed to a test compound or to vehicle controls. After exposure, the cells can be assayed to measure the expression and/or activity of components of the signal transduction pathway of the melanocortin receptor. Alternatively, the activity of the signal transduction pathway itself can be assayed. For example, after exposure, cell lysates can be assayed for induction of cAMP. The ability of a test compound to increase levels of cAMP above those levels seen with cells created with a vehicle control and, preferably, compared to an otherwise identical cell which does not express MC4-R, indicates that the test compound induces signal transduction mediated by the melanocortin receptor expressed by the host cell.

To determine intracellular cAMP concentrations, a scintillation proximity assay (SPA) may be utilized (SPA kit is provided by Amersham Life Sciences, Illinois). The assay utilizes ¹²⁵I labeled cAMP, an anti-cAMP antibody, and a scintillant-incorporated microsphere coated with a secondary antibody. When brought into close proximity to the microsphere through the labeled cAMP-antibody complex, ¹²⁵I will excite the scintillant to emit light. Unlabeled cAMP extracted from cells competes with the ¹²⁵I-labeled cAMP for binding to the antibody and thereby diminishes scintillation. The assay may be performed in 96-well plates to enable high-throughput screening and 96 well-based scintillation counting instruments such as those manufactured by Wallac or PAckard may be used for readout.

In screening for compounds that may act as antagonists of MC4-R using receptor activity assays, it is necessary to include ligands that activate the MC4-R, e.g., (α-MSH, (β-MSH, agouti, ARP or ACTU, to test for inhibition of signal transduction by the test compound as compared to vehicle controls.

In a specific embodiment of the invention, constructs containing the CAMP responsive element linked to any of a variety of different reporter genes may be introduced into cells expressing the melanocortin receptor. Such reporter genes may include but is not limited to chloramphenicol acetyltransferase (CAT), luciferase, GUS, growth hormone, or placental alkaline phosphatase (SEAP). Following exposure of the cells to the test compound, the level of reporter gene expression may be quantitated to determine the test compound's ability to regulate receptor activity. Alkaline phosphatase assays are particularly useful in the practice of the invention as the enzyme is secreted from the cell. Therefore, tissue culture supernatant may be assayed for secreted alkaline phosphatase. In addition, alkaline phosphatase activity may be measured by calorimetric, bioluminescent or chemilumenscent assays such as those described in Bronstein et al. (1994, Biotechniques 17:172-177). Such assays provide a simple, sensitive easily automatable detection system for pharmaceutical screening.

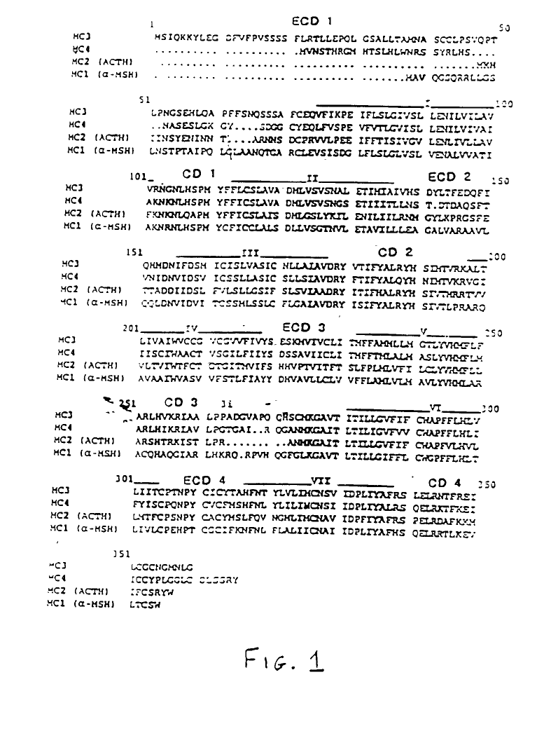

When it is desired to discriminate between the melanocortin receptors and to identify compounds that selectively agonize or antagonize the MC4-R, the assays described above should be conducted using a panel of host cells, each genetically engineered to express one of the melanocortin receptors (MC1-R through MC5-R). Expression of the human melanocortin receptors is preferred for drug discovery purposes. To this end, host cells can be genetically engineered to express any of the amino acid sequences shown for melanocortin receptors 1 through 5 in FIG. 1. The cloning and characterization of each receptor has been described: MC1-R and MC2-R (Mountjoy, 1992, Science 257: 1248-1251; Chhajlani & Wikberg, 1992 FEBS Lett. 309: 417-420); MC3-R (Roselli-Rehfuss et al., 1993, Proc. Natl. Acad. Sci., USA 90: 8856-8860; Gantz et al., 1993, J. Biol. Chem. 268: 8246-8250); MC4-R (Gantz et al., 1993, J. Biol. Chem. 268: 15174-15179; Mountjoy et al., 1994, Mol. Endo. 8: 1298-1308); and MC5-R (Chhajlani et al., 1993, Biochem. Biophys. Res. Commun. 195: 866-873; Gantz et al., 1994, Biochem. Biophys. Res. Commun. 200; 1214-1220), each of which is incorporated by reference herein in its entirety. Thus, each of the foregoing sequences can be utilized to engineer a cell or cell line that expresses one of the melanocortin receptors for use in screening assays described herein. To identify compounds that specifically or selectively regulate MC4-R activity, the activation or inhibition of MC4-R activation is compared to the effect of the test compound on the other melanocortin receptors.

In a specific embodiment, MC1-R through MC5-R cDNAs are expressed in 293 cells under the transcriptional control of the CMV promoter. Stable cell lines are established. Because transfected human MC2-R (ACTH-R) did not express very well in 293 cells, the human adrenocortical carcinoma cell line H295 (ATCC No. CRL-2128), which expresses endogenous ACTH-R, may be used in screening assays in addition to a stable cell line that expresses transfected ACTH-R. In the first round of screening, the MC4-R expressing cell line is used to identify candidate compounds that activated the MC4-R. Once identified, those candidate compounds can be tested to determine whether they selectively activate the MC4-R. The activation of the melanocortin receptors may be assayed using, for example, the SPA assay described above.

Alternatively, if the host cells express more than one melanocortin peptide receptor, the background signal produced by these receptors in response to melanocortin peptides must be “subtracted” from the signal (see Gantz et al., supra). The background response produced by these non-MC4-R melanocortin receptors can be determined by a number of methods, including elimination of MC4-R activity by antisense, antibody or antagonist. In this regard, it should be noted that wild type CHO cells demonstrate a small endogenous response to melanocortin peptides which must be subtracted from background. Alternatively, activity contributed from other melanocortin receptors could be eliminated by activating host cells with a MC4-R-specific ligand, or including specific inhibitors of the other melanocortin receptors.

### Non-Cell Based Assays

In addition to cell based assays, non-cell based assay systems may be used to identify compounds that interact with, e.g., bind to MC4-R. Such compounds may act as antagonists of MC4-R activity and may be used in the treatment of addictive behavior disorders.

Since MC4-R is a G protein coupled receptor having seven transmembrane domains, isolated membranes may be used to identify compounds that interact with MC4-R. For example, in a typical experiment using isolated membranes, 293 cells may be genetically engineered to express the MC4-R. Membranes can be harvested by standard techniques and used in an in vitro binding assay. ¹²⁵I-labelled ligand (e.g., ¹²⁵I-labelled α-MSH, β-MSH, or ACTH) is bound to the membranes and assayed for specific activity; specific binding is determined by comparison with binding assays performed in the presence of excess unlabelled ligand.

To identify MC4-R ligands, membranes are incubated with labeled ligand in the presence or absence of test compound. Compounds that bind to the receptor and compete with labeled ligand for binding to the membranes reduced the signal compared to the vehicle control samples.

Alternatively, soluble MC4-R may be recombinantly expressed and utilized in non-cell based assays to identify compounds that bind to MC4-R. As described above, the recombinantly expressed MC4-R polypeptides or fusion proteins containing one or more of the ECDs of MC4-R can be used in the non-cell based screening assays. Alternatively, peptides corresponding to one or more of the CDs of MC4-R, or fusion proteins containing one or more of the CDs of MC4-R can be used in non-cell based assay systems to identify compounds that bind to the cytoplasmic portion of the MC4-R; such compounds may be useful to antagonize the signal transduction pathway of the MC4-R. In non-cell based assays the recombinantly expressed MC4-R can be attached to a solid substrate such as a test tube, microtitre well or a column, by means well known to those in the art. The test compounds are then assayed for their ability to bind to the MC4-R.

The MC4-R protein and MC4-R ligand used in the present invention can be used in a variety of forms. The proteins can be used in a highly purified form, free of naturally occurring contaminants. Alternatively, a crude preparation containing a mixture of cellular components as well as the target protein can be used. So long as the association of the MC4-R protein with the compound to be tested and/or the MC4-R ligand can be identified in the sample, the MC4-R protein and MC4-R ligand are in a suitable form for use in the above described assay. Additionally, she MC4-R protein and/or the MC4-R ligand can be modified to contain a detectable label/signal generation system to facilitate detection. Methods for attaching compounds such as fluorescence tags and secondary labeling compounds such as biotin, are well known in the art.

As indicated above, direct binding to the MC4-R protein or the MC4-R ligand can be used as first step in identifying compounds that antagonize the MC4-R protein. For example, in such methods, compounds are first screened for the ability to bind to either the MC4-R protein or the MC4-R ligand. Compounds that bind MC4-R or the ligand are then screened for the ability to block ligand/receptor interaction, antagonize the MC4-R receptor in an activity assay or the ability to ameliorate an addictive behavior in an animal model, or a combination thereof.

In one aspect of the invention the screens may be designed to identify compounds that antagonize the interaction between MC4-R and MC4-R ligands such as α-MSH, βMSH and ACTH. In such screens, the MC4-R ligands are labeled and test compounds can be assayed for their ability to antagonize the binding of labeled ligand to MC4-R.

Such peptides, polypeptides, and fusion proteins can be prepared by recombinant DNA techniques. For example, nucleotide sequences encoding one or more of the four domains of the ECD of the serpentine MC4-R can be synthesized or cloned and ligated together to encode a soluble ECD of the MC4-R. The DNA sequence encoding one or more of the four ECDs (ECD1-4 in FIG. 1) can be ligated together directly or via a linker oligonucleotide that encodes a peptide spacer. Such linkers may encode flexible, glycine-rich amino acid sequences thereby allowing the domains that are strung together to assume a conformation that can bind MC4-R ligands. Alternatively, nucleotide sequences encoding individual domains within the ECD can be used to express MC4-R peptides. In addition, mutant MC4-R proteins can be expressed by recombinant DNA techniques.

A variety of host-expression vector systems may be utilized to express nucleotide sequences encoding the appropriate regions of the MC4-R to produce such polypeptides. Where the resulting peptide or polypeptide is a soluble derivative (e.g., peptides corresponding to the ECDs; truncated or deleted in which the TMs and/or CDs are deleted) the peptide or polypeptide can be recovered from the culture media. Where the polypeptide or protein is not secreted, the MC4-R product can be recovered from the host cell itself.

The host-expression vector systems also encompass engineered host cells that express the MC4-R or functional equivalents in situ, i.e., anchored in the cell membrane. Purification or enrichment of the MC4-R from such expression systems can be accomplished using appropriate detergents and lipid micelles and methods well known to those skilled in the art. However, such engineered host cells themselves may be used in situations where it is important not only to retain the structural and functional characteristics of the MC4-R, but to assess biological activity, e.g., in drug screening assays.

The host-expression vector systems that may be used for purposes of the invention include but are not limited to microorganisms such as bacteria (e.g., E. coli and B. subtilis) transformed with recombinant bacteriophage DNA, plasmid DNA or cosmid DNA expression vectors containing MC4-R nucleotide sequences; yeast (e.g., Saccharonyces and Pichia) transformed with recombinant yeast expression vectors containing the MC4-R nucleotide sequences; insect cell systems infected with recombinant virus expression vectors (e.g., baculovirus) containing the MC4-R sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing MC4-R nucleotide sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the MC4-R gene product being expressed. For example, when a large quantity of such a protein is to be produced, for the generation of pharmaceutical compositions of MC4-R protein or for raising antibodies to the MC4-R protein, for example, vectors that direct the expression of high levels of fusion protein products that are readily purified may be desirable. Such vectors include, but are not limited, to the E. coli expression vector pUR278 (Ruther et al., 1983, EMBO J. 2:1791), in which the MC4-R coding sequence may be ligated individually into the vector in frame with the lacZ coding region so that a fusion protein is produced; pIN vectors (Inouye & Inouye, 1985, Nucleic Acids Res. 13:3101-3109; Van Heeke & Schuster, 1989, J. Biol. Chem. 264:5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The PGEX vectors are designed to include thrombin or factor Xa protease cleavage sites so that the cloned target gene product can be released from the GST moiety.

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht et al., 1991, Proc. Natl. Acad. Sci. USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni2+(nitriloacetic acid-agarose columns and histidine-tagged proteins are selectively eluted with imidazole-containing buffers.

In an insect system, Autographa californica nuclear polyhedrosis virus (AcNPV) is used as a vector to express foreign genes. The virus grows in Spodoptera frugiperda cells. The MC4-R ceding sequence may be cloned individually into non-essential regions (for example the polyhedrin gene) of the virus and placed under control of an AcNPV promoter (for example the polyhedrin promoter). Successful insertion of MC4-R gene coding sequence will result in inactivation of the polyhedrin gene and production of non-occluded recombinant virus (i.e., virus lacking the proteinaceous coat coded for by the polyhedrin gene). The recombinant viruses are then used to infect cells in which the inserted gene is expressed. (e.g., see Smith et al., 1983, J. Virol. 46: 584; Smith, U.S. Pat. No. 4,215,051).

In mammalian host cells, a number of viral-based expression systems may be utilized. In cases where an adenovirus is used as an expression vector, the MC4-R nucleotide sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the MC4-R gene product in infected hosts. (e.g., See Logan & Shenk, 1984, Proc. Natl. Acad. Sci. USA 81:3655-3659). Specific initiation signals may also be required for efficient translation of inserted MC4-R nucleotide sequences. These signals include the ATG initiation codon and adjacent sequences. In cases where an entire MC4-R gene or cDNA, including its own initiation codon and adjacent sequences, is inserted into the appropriate expression vector, no additional translational control signals may be needed. However, in cases where only a portion of the MC4-R coding sequence is inserted, exogenous translational control signals, including, perhaps, the ATG initiation codon, must be provided. Furthermore, the initiation codon must be in frame with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (See Bittner et al., 1987, Methods in Enzymol. 153:516-544).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. Accordingly, eukaryotic host cells that possess the cellular machinery for proper processing of the primary transcript, glycosylation, and phosphorylation of the gene product may be used. Such mammalian host cells include, but are not limited to, CHO, VERO, BHK, HeLa, COS, LMDCK, 293, 3T3 and WI38 cell lines.

For long-term, high-yield production of recombinant proteins, stable expression is preferred. For example, cell lines that stably express the MC4-R sequences described above may be engineered. Rather than using expression vectors that contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines that express the MC4-R gene product. Such engineered cell lines may be particularly useful in screening and evaluation of compounds that affect the endogenous activity of the MC4-R gene product.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22:817) genes can be employed in tk-, hgprt- or aprt- cells, respectively. Also, antimetabolite resistance can be used as the basis of selection for the following genes: dhfr, which confers resistance to methotrexate (Wigler et al., 1980, Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147).

Assays For Compounds or Compositions that Antagonize Expression of the MC4-R

In addition to assays based on MC4-R protein activity or binding, in vitro cell based assays may be designed to screen for compounds that regulate MC4-R expression at either the transcriptional or translational level. Antagonist of the transcription or translation of MC4-R can be used as antagonists of the MC4-R protein e.g. by reducing the amount of MC4-R protein produce by a subject.

In one embodiment, DNA encoding a reporter molecule can be linked to a regulatory element of the MC4-R gene and used in appropriate intact cells, cell extracts or lysates to identify compounds that antagonize MC4-R gene expression.

Appropriate cells or cell extracts are prepared from any cell type that normally expresses the MC4-R gene, thereby ensuring that the cell extracts contain the transcription factors required for in vitro or in vivo transcription. The screen can be used to identify compounds that antagonize the expression of the reporter construct. In such screens, the level of reporter gene expression is determined in the presence of the test compound and compared to the level of expression in the absence of the test compound.

To identify compounds that regulate MC4-R translation, cells or in vitro cell lysates containing MC4-R transcripts may be rested for modulation of MC4-R mRNA translation. To assay for inhibitors of MC4-R translation, test compounds are assayed for their ability to antagonize the translation of MC4-R mRNA in in vitro translation extracts.

Compounds that decrease the level of MC4-R expression, either at the transcriptional or translational level, are useful for treatment of addictive behavior disorders.

Compounds identified via assays such as those described herein may be useful, for example, in elaborating the biological function of the MC4-R gene product, and for ameliorating addictive behavior disorders. Assays for testing the efficacy of compounds identified in the cellular screen can be tested in art known animal model systems, such as those employed in the Examples, for addictive behavior disorders. For example, animal models may be exposed to a compound, suspected of exhibiting an ability to ameliorate addictive behavior, at a sufficient concentration and for a time sufficient to elicit such an amelioration of addictive behavior in the exposed animals. The response of the animals to the exposure may be monitored by assessing the reversal of the addictive behavior. With regard to intervention, any treatments that reverse any aspect of addictive behavior-like symptoms should be considered as candidates for human addictive behavior disorder therapeutic intervention.

Uses for Compounds that Antagonize the MC4-R Protein

The methods of the present invention are suitable for use in identifying compounds for treating addiction to a wide variety of addictive compounds. Repeated administration to a subject of certain drugs such as cocaine, opiates, alcohol, hallucinogens, minor tranquilizers, nicotine, and stimulants can lead to physical and/or psychological dependence upon that drug or substance. Although almost any drug is capable of addiction, certain drugs demonstrated a marked propensity to become addictive. These include opiates (opium, morphine, heroin, and so called “designer drugs,” which are opiates that have been chemically modified to avoid literal violation of the controlled substance laws, or to create a better or different psychophysiological effect), methadone, cocaine, nicotine, alcohol, certain depressants, and certain stimulants. When the drug or substance of abuse is withdrawn from a dependent subject, the subject develops certain symptoms including sleep and mood disturbance and intense craving for the drug or substance of abuse. These symptoms may be collectively described as a withdrawal syndrome in connection with the present invention. As discussed in the Background, many drugs of addiction have been found to stimulate similar behavior centers of the brain. In the Examples, data show that two classes of addictive compounds, cocaine and opiates, stimulate similar biological response. Accordingly, compounds identified in the present methods will likely be useful in treating addictive behavior to a wide variety of addictive stimulus.

As used herein, the term “addictive disease, disorder, behavior or addiction” refers to a disease or disorder in which the subject has an extreme craving or compulsion to repeat a particular behavior. The present invention is particularly directed to therapeutic treatment of a drug addiction. Notwithstanding the specifically exemplified ability of the present invention to modulate the biochemistry and behavior that correlate with drug abuse, the invention further extends to the treatment of addictive psychological diseases or disorders, such as, but not limited to, obsessive-compulsive disease.

The methods of the present invention, and the compounds identified using the present methods, are use to treat or prevent an addictive disease or disorder in a subject. Preferably the subject is a human, however, as animals in addition to humans may demonstrate addictive diseases or disorders, whether resulting from addiction to opiates or other drugs subsequent to a veterinary procedure or as a result of a psychological disorder, such as an obsessive compulsive-type of disorder, the invention can be used in birds, such as chickens, turkeys, and pets; in mammals, including but not limited to domesticated animals (canine and feline); farm animals (bovine, ovine, equine, caprine, porcine, and the like); rodents; and undomesticated animals.

Administration of Compounds Identified Using the Present Invention

The compounds of the present invention can be provided alone, or in combination with another compound that modulates an addictive behavior. For example, a compound of the present invention used to that reduce heroin addiction can be administered in combination with other anti-addictive compounds. As used herein, two compounds are said to be administered in combination when the two compounds are administered simultaneously or are administered independently in a fashion such that the compounds will act at the same time.

The compounds identified using the methods of the present invention can be administered via parenteral, subcutaneous, intravenous, intramuscular, intraperitoneal, transdermal, or buccal routes. Alternatively, or concurrently, administration may be by the oral route. The dosage administered will be dependent upon the age, health, and weight of the recipient, kind of concurrent treatment, if any, frequency of treatment, and the nature of the effect desired.

The present invention further provides compositions containing one or more compounds identified using the present invention, in a pharmaceutically acceptable form. While individual needs vary, determination of optimal ranges of effective amounts of each component is within the skill of the art. Typical dosages comprise 0.1 to 100 mg/kg/body wt. The preferred dosages comprise 1 to 100 mg/kg/body wt. The most preferred dosages comprise 10 to 100 mg/kg/body wt.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for Injectable solutions. Suitable pharmaceutical carriers are described in “Remington's Pharmaceutical Sciences” by E. W. Martin.

The phrase “therapeutically effective amount” is used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition in the host. Preferably, the activity so modified or modulated according to the invention is a behavioral activity. In particular, the behavioral activity may be locomotor activity, conditioned place preference, or drug self-administration.

Without further description, it is believed that one of ordinary skill in the art can, using the preceding description and the following illustrative examples, make and utilize the compounds of the present invention and practice the claimed methods. The following working examples therefore, specifically point out preferred embodiments of the present Invention, and are not to be construed as limiting in any way the remainder of the disclosure.

### EXAMPLES

The Role of MC4-R in the Regulation of Addictive Behavior

The specific role of the MC4-R protein in vivo was investigated by:

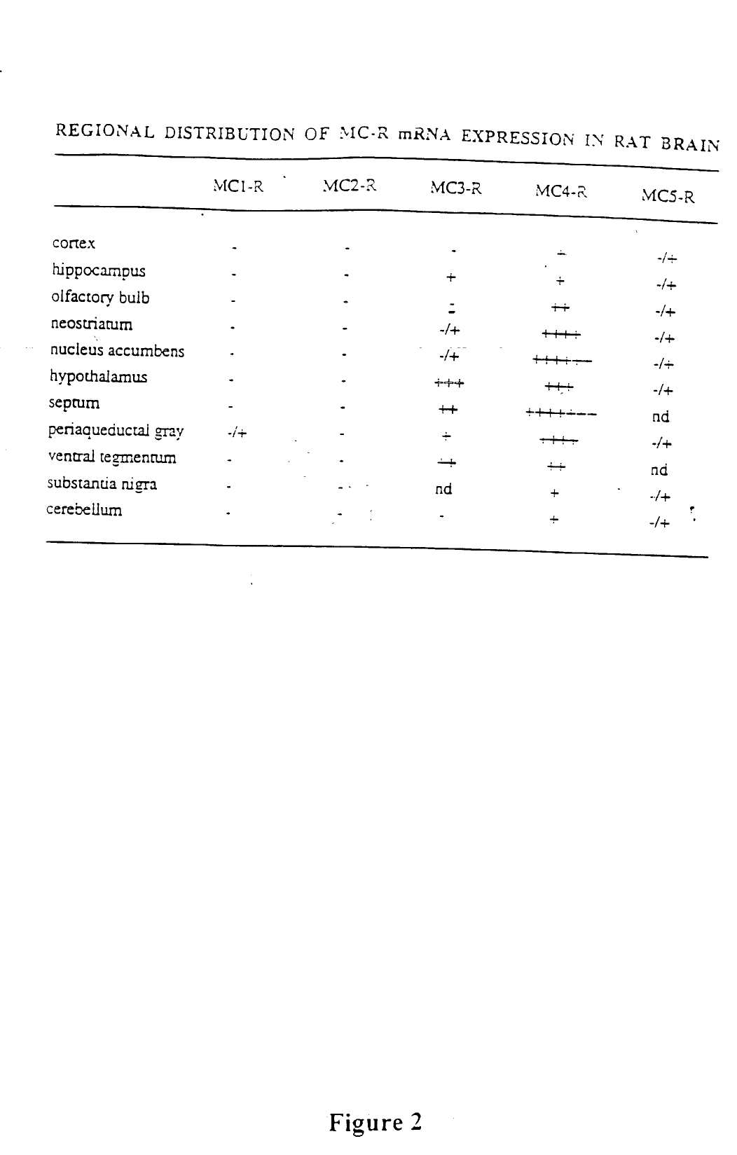

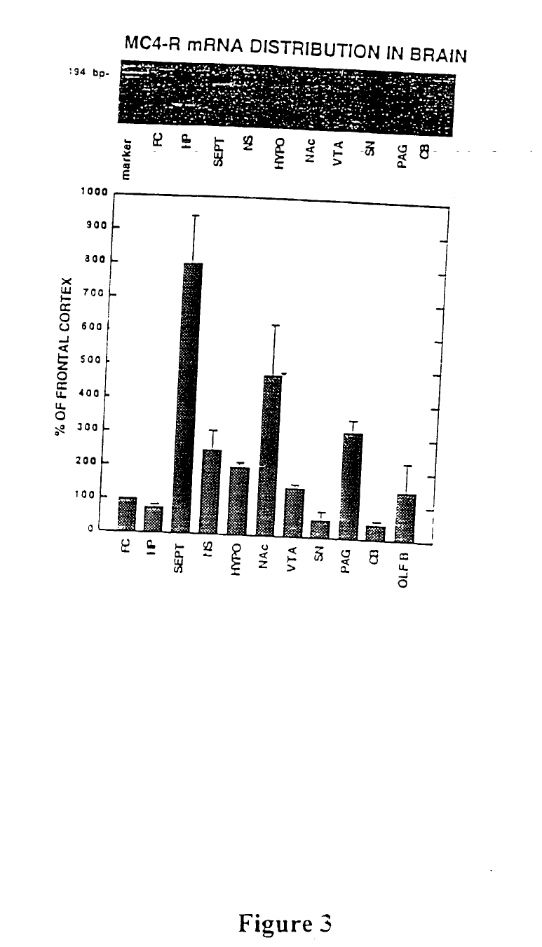

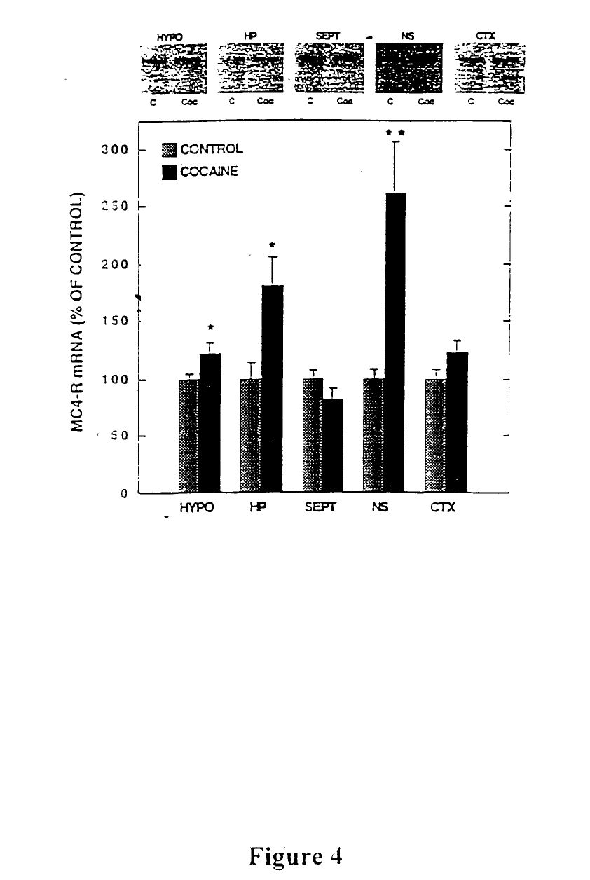

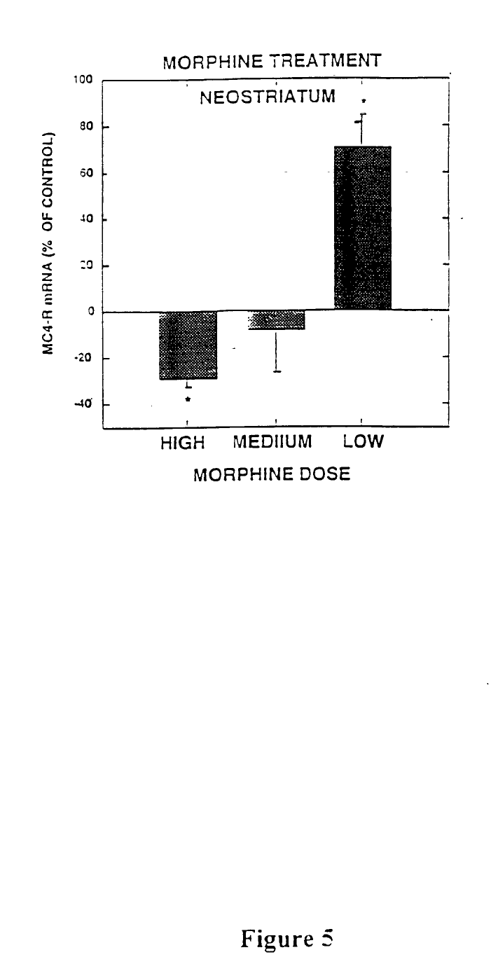

examining expression patterns of the MC-R family of proteins in rat brain regions involved in addictive behavior (FIGS. 2 and 3);

examining the changes in mRNA expression levels of rat MC4-R in response to cocaine or morphine administration (FIGS. 4 and 5);

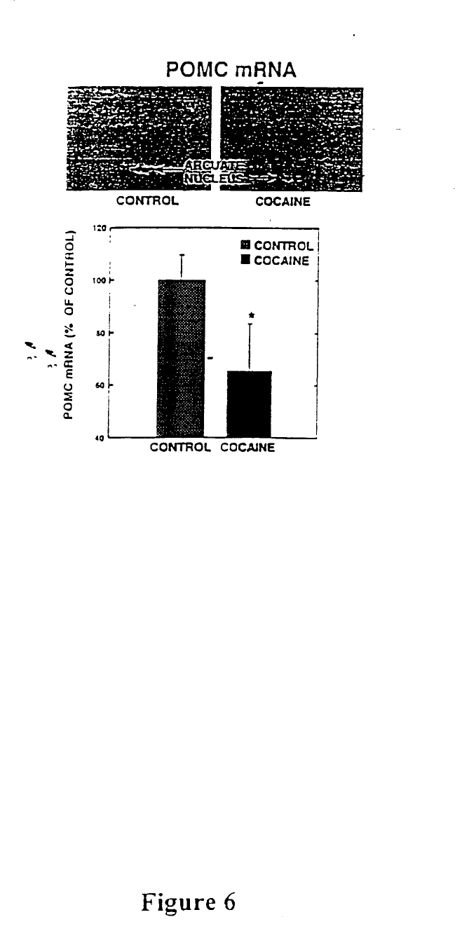

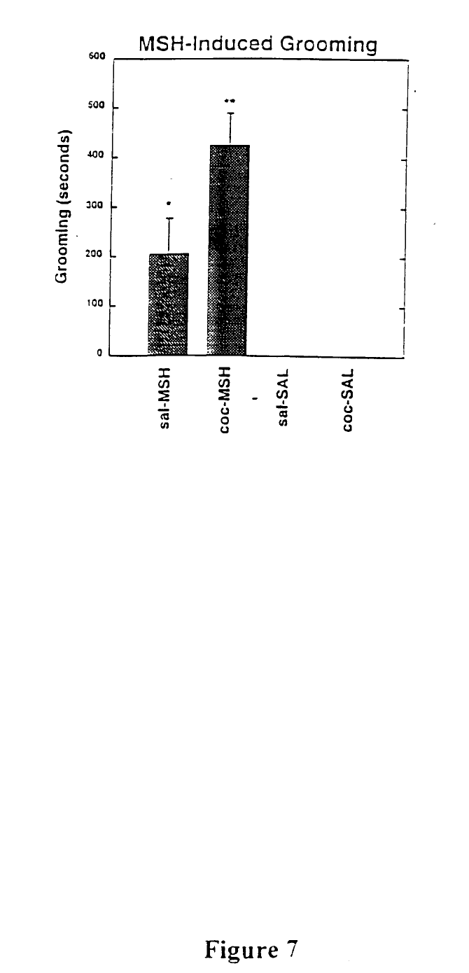

examining the changes in mRNA expression levels of POMC in response to cocaine administration (FIG. 6); examining the changes seen in MSH-Induced grooming behavior in rats caused by the administration of combinations of saline, MSH, and cocaine (FIG. 7);

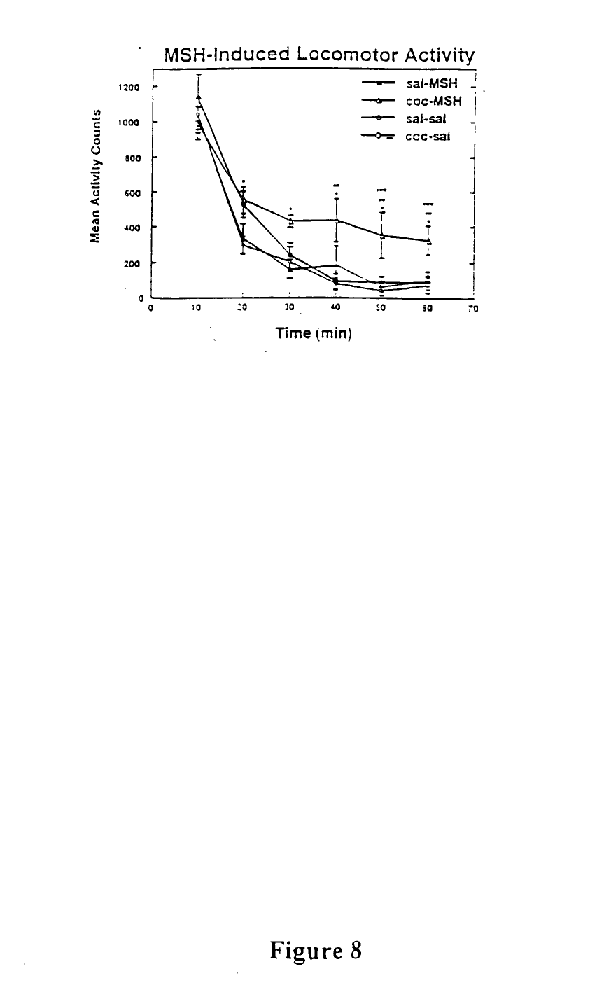

examining changes seen in MSH-induced locomotor activity in rats caused by the administration of combinations of saline, MSH, and cocaine (FIG. 8);

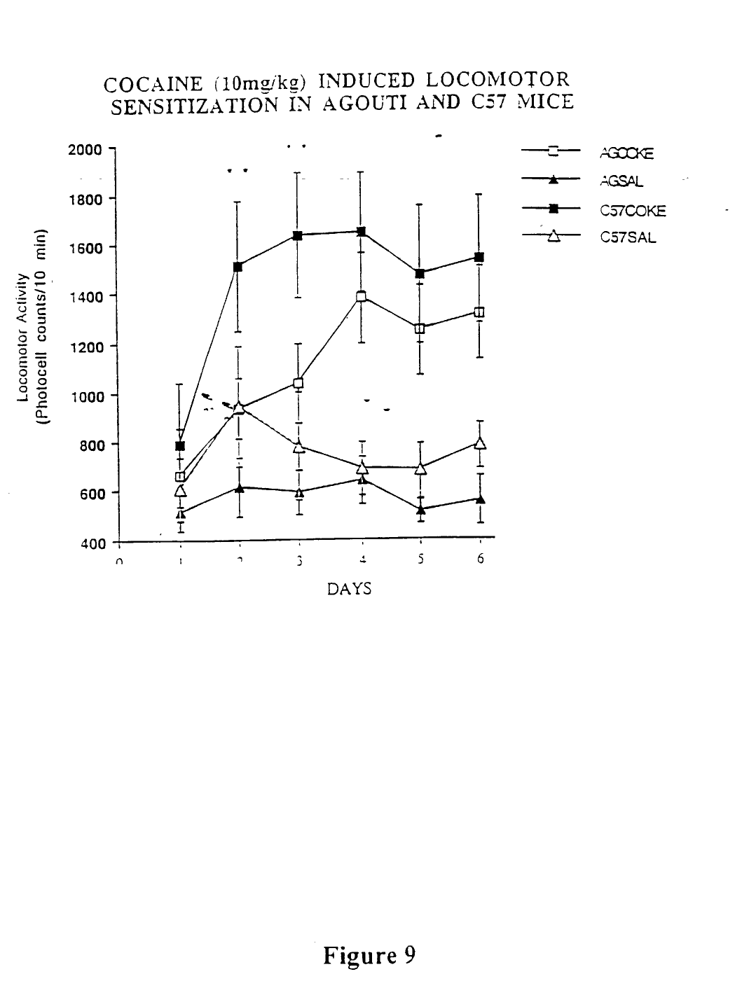

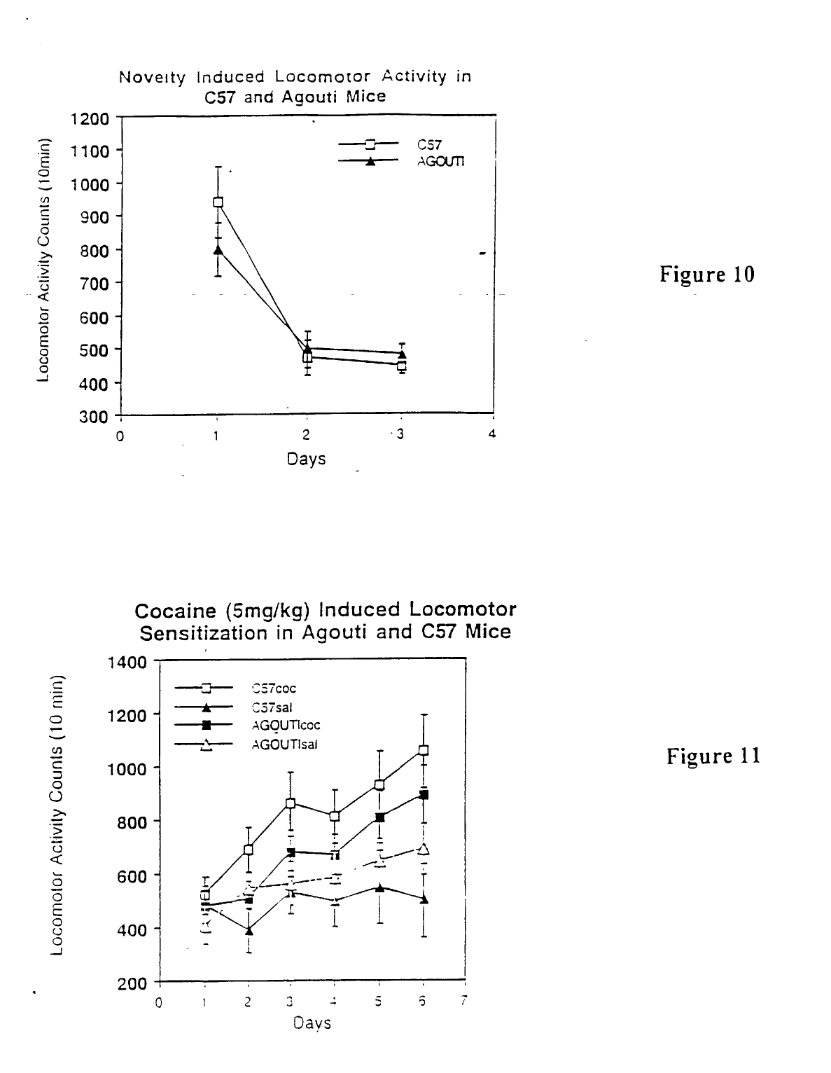

examining cocaine (10 mg/kg) induced locomotor sensitization in Agouti and C57 mice (FIG. 9); examining cocaine (5 mg/kg) induced locomotor sensitization in Agouti and C57 mice (FIG. 11);.

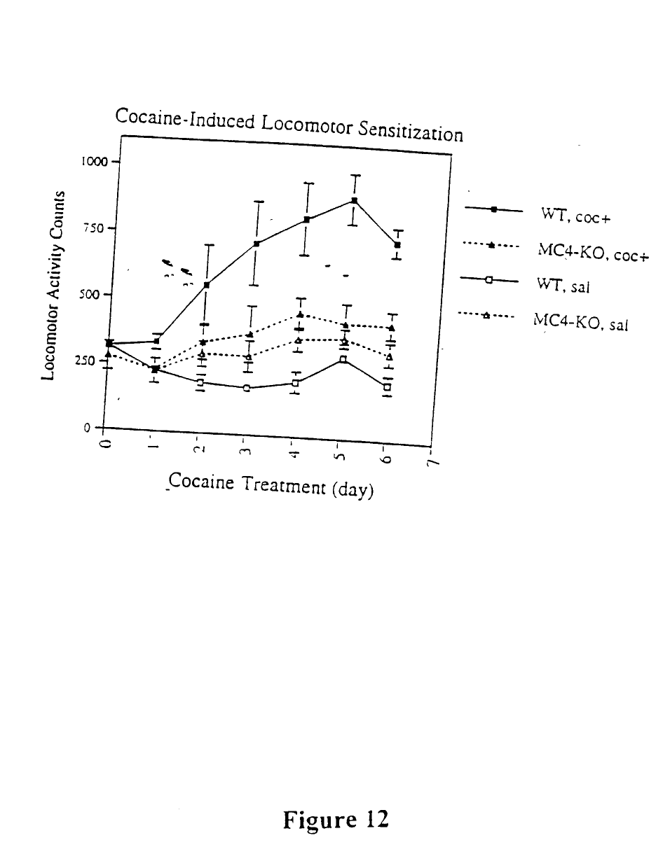

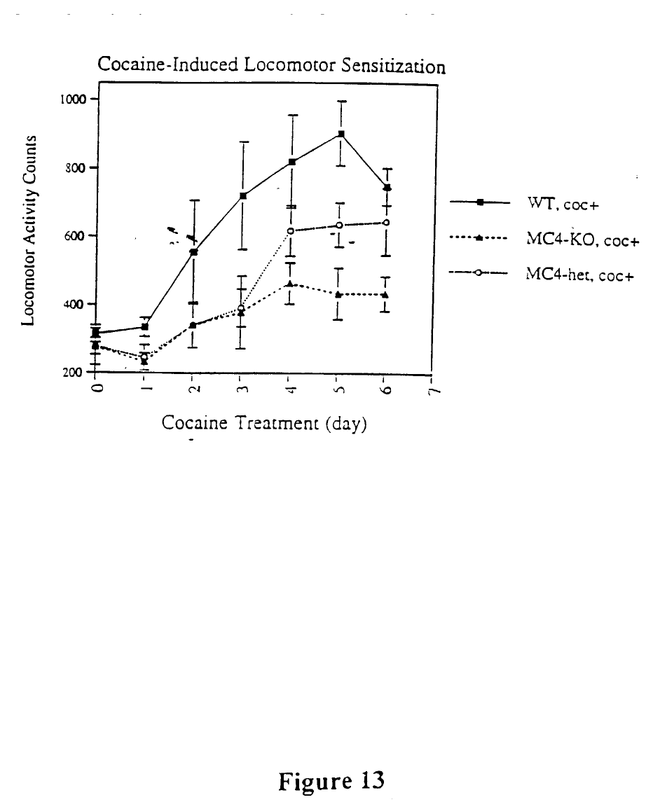

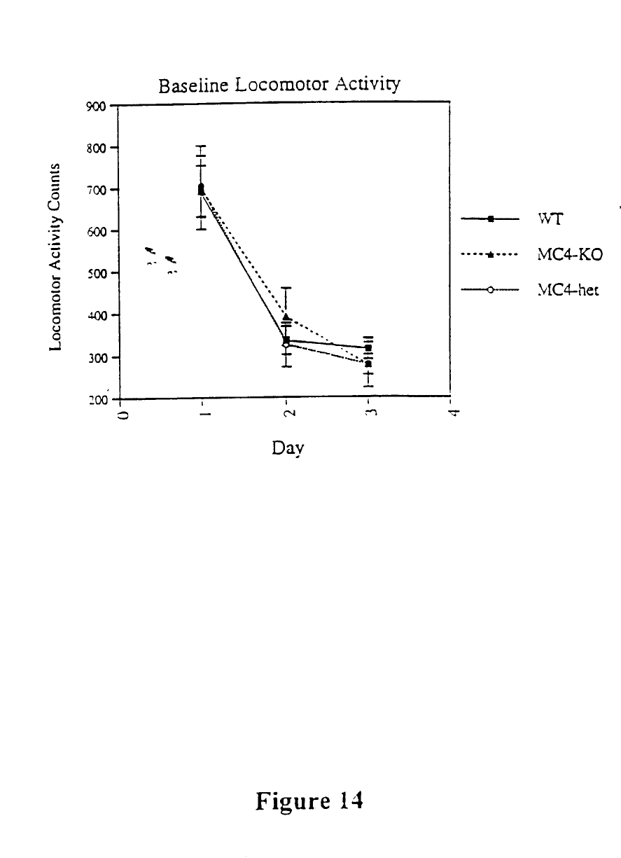

examining cocaine (10 mg/kg) induced locomotor sensitization in C57 (wild-type) and MC4-R knockout (homozygous and heterozygous) mice (FIGS. 12, 13 and 14);

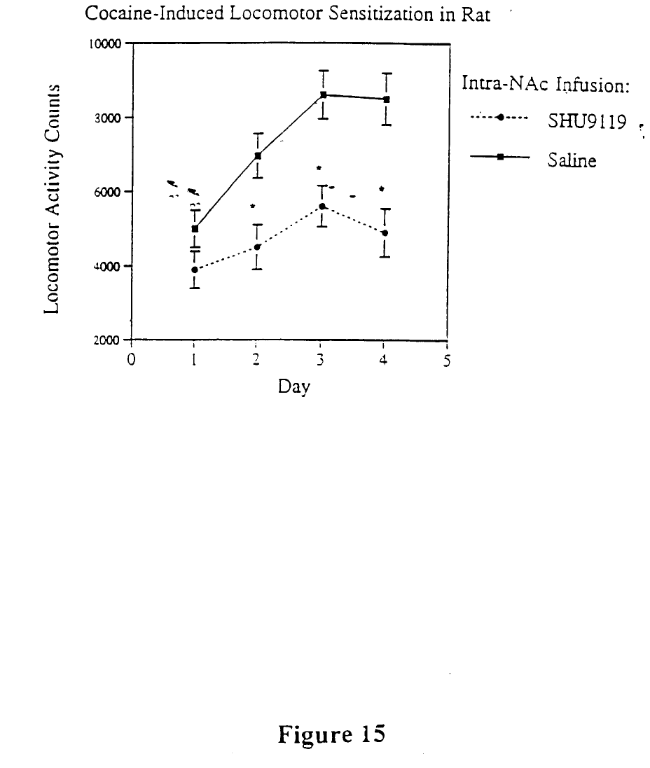

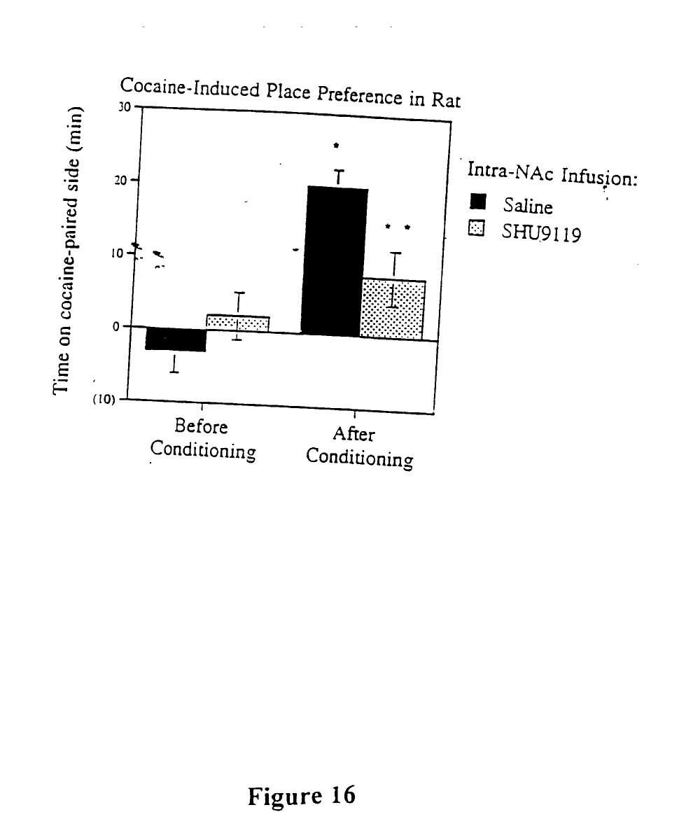

examining cocaine (10 mg/kg) induced locomotor sensitization in Rats treated with and without Intra-Nac infusions of SHU9119 (an antagonist of MC4-R) (FIG. 15); and

examining cocaine induced place preference in Rats treated with and without Intra-Nac infusions of SHU9119 (an antagonist of MC4-R7.

All of the results obtained indicate that MC4-R is involved in mediating addictive behavior, particularly in response to cocaine or morphine addiction, and that antagonizing MC4-R results in decreased addictive behavior.

What is claimed is:

1. A method for identifying compounds that regulate addictive behavior, comprising:
a) contacting a test compound with a melanocortin 4-receptor (MC4-R),
b) determining whether the test compound binds to said MC4-R,
c) administering a compound identified as binding to said MC4-R in step (b) to an animal,
d) determining whether said compound reduces an addictive behavior, and
e) selecting a compound that reduces an addictive behavior in step (d).

2. A method for identifying compounds that regulate addictive behavior, comprising:
a) contacting a melanocortin peptide in the presence and absence of a test compound with a melanocortin 4-receptor,
b) determining whether the test compound inhibits the binding of the melanocortin peptide to the melanocortin 4-receptor,
c) administering a compound identified as inhibiting binding of a melanocortin peptide to said MC4-R in step (b) to an animal,
d) determining whether said compound reduces an addictive behavior, and
e) selecting a compound that reduces an addictive behavior in step (d).

3. The method of claim 1, wherein said MC4-R is expressed on the surface of a recombinant cell.

4. The method of claim 2, wherein said MC4-R is expressed on the surface of a recombinant cell.

5. The method of claim 3, wherein said recombinant cell is an eukaryotic cell.

6. The method of claim 4, wherein said recombinant cell is an eukaryotic cell.

7. The method of claim 4 wherein the inhibition of the binding of the melanocortin peptide to the MC4-R is determined by measuring induction of cAMP in said recombinant cell.

8. The method of claim 6 wherein the inhibition of the binding of the melanocortin peptide to the MC4-R is determined by measuring induction of cAMP in said recombinant cell.

9. The method of claim 7 in which the cell further contains a reporter gene operatively associated with a cAMP responsive element, and induction of cAMP is indicated by expression of the reporter gene.

10. The method of claim 8 in which the cell further contains a reporter gene operatively associated with a cAMP responsive element, and induction of cAMP is indicated by expression of the reporter gene.

11. The method of claim 9 in which the reporter gene is alkaline phosphatase, chloramphenicol acetyltransferase, luciferase, glucuronide synthetase, growth hormone, or placental alkaline phosphatase.

12. The method of claim 10 in which the reporter gene is alkaline phosphatase, chloramphenicol acetyltransferase, luciferase, glucuronide synthetase, growth hormone, or placental alkaline phosphatase.

13. A method for the treatment of addictive behavior disorders, comprising administering an effective amount of a compound that antagonizes the activity of the melanocortin 4-receptor.

14. A pharmaceutical formulation for the treatment of addictive behavior disorders, comprising a compound that antagonizes the melanocortin 4-receptor, mixed with a pharmaceutically acceptable carrier.


SEQUENCE LISTING (4 sequences)

SEQ ID NO: 1 - protein, 317 aa, Homo sapiens
MAVQGSQRRLLGSLNSTPTAIPQLGLAANQTGARCLEVSISDGLFLSLGLVSLVENALVVATIAKNRNLHSPMYCFICCLALSDLLVSGTNVLETAVILLLEAGALVARAAVLQQLDNVIDVITCSSMLSSLCFLGAIAVDRYISIFYALRYHSIVTLPRAPRAVAAIWVASVVFSTLFIAYYDHVAVLLCLVVFFLAMLVLMAVLYVHMLARACQHAQGIARLHKRGRPVHQGFGLKGAVTLTILLGIFFLCWGPFFLHLTLIVLCPEHPTCGCIFKNFNLFLALIICNAIIDPLIYAFHSQELRRTLKEVLTCSW

SEQ ID NO: 2 - protein, 297 aa, Homo sapiens
MKHIINSYENINNTARNNSDCPRVVLPEEIFFTISIVGVLENLIVLLAVFKNKNLQAPMYFFICSLAISDMLGSLYKILENILIILRNMGYLKPRGSFETTADDIIDSLFVLSLLGSIFSLSVIAADRYITIFHALRYHSIVTMRRTVVVLTVIWTFCTGTGITMVIFSHHVPTVITFTSLFPLMLVFILCLYVHMFLLARSHTRKISTLPRANMKGAITLTILLGVFIFCWAPFVLHVLLMTFCPSNPYCACYMSLFQVNGMLIMCNAVIDPFIYAFRSPELRDAFKKMIFCSRYW

SEQ ID NO: 3 - protein, 360 aa, Homo sapiens
MSIQKKYLEGDFVFPVSSSSFLRTLLEPQLGSALLTAMNASCCLPSVQPTLPNGSEHLQAPFFSNQSSSAFCEQVFIKPEIFLSLGIVSLLENILVILAVVRNGNLHSPMYFFLCSLAVADMLVSVSNALETIMIAIVHSDYLTFEDQFIQHMDNIFDSMICISLVASICNLLAIAVDRYVTIFYALRYHSIMTVRKALTLIVAIWVCCGVCGVVFIVYSESKMVIVCLITMFFAMMLLMGTLYVHMFLFARLHVKRIAALPPADGVAPGGHSCMKGAVTITILLGVFIFCWAPFFLHLVLIITCPTNPYCICYTAHFNTYLVLIMCNSVIDPLIYAFRSLELRNTFREILCGCNGMNLG

SEQ ID NO: 4 - protein, 332 aa, Homo sapiens [sequence omitted]